(19) United States
(12) Patent Application Publication
(10) Pub. No.: US 2022/0110510 A1
(43) Pub. Date: Apr. 14, 2022

(54) ENDOSCOPE APPARATUS, CONTROL METHOD, CONTROL PROGRAM, AND ENDOSCOPE SYSTEM

(71) Applicant: FUJIFILM Corporation, Tokyo (JP)

(72) Inventors: Mio AZUMA, Ashigarakami-gun (JP); Kenichi SHIDARA, Ashigarakami-gun (JP)

(73) Assignee: FUJIFILM Corporation, Tokyo (JP)

(21) Appl. No.: 17/560,589

(22) Filed: Dec. 23, 2021

Related U.S. Application Data
(63) Continuation of application No. PCT/JP2020/018916, filed on May 12, 2020.

(30) Foreign Application Priority Data
Aug. 9, 2019 (JP) 2019-147056

(51) Int. Cl. A61B 1/045; A61B 1/018; A61B 1/00

(52) U.S. Cl. CPC A61B 1/045; A61B 1/018; A61B 1/00006; A61B 1/00009; A61B 1/00013; A61B 1/00043

(57) ABSTRACT

An endoscope has an insertion part to be inserted into a living body and an operating part. An endoscope apparatus is connected to the endoscope. The control unit displays an image associated with an operation capable of being executed by the endoscope apparatus on a part of a display screen of a display device. A visual line position detection unit detects a visual line position of an operator of the endoscope. The control unit determines whether or not the operator gazes at the image based on the visual line position detected by the visual line position detection unit. Moreover, the control unit executes the operation associated with the image based on a result of the determination and a command signal input in response to a manipulation by the operator with respect to the operating part.

### CROSS-REFERENCE TO RELATED APPLICATIONS

This application is a Continuation of PCT International Application No. PCT/JP2020/018916 filed on May 12, 2020, which claims priority under 35 U.S.C § 119(a) to Japanese Patent Application No. 2019-147056 filed on Aug. 9, 2019. Each of the above application(s) is hereby expressly incorporated by reference, in its entirety, into the present application.

### BACKGROUND OF THE INVENTION

### 1. Field of the Invention

The present invention relates to an endoscope apparatus, a control method, a control program, and an endoscope system.

### 2. Description of the Related Art

In the related art, in an endoscope apparatus that performs a control of displaying an image obtained by an endoscope, a configuration for receiving various manipulations, such as a manipulation of switching a display mode of the image, has been proposed. For example, in JP1999-332883A (JP-H11-332883A), as a configuration for manipulating an endoscope or a medical apparatus relating to the endoscope, a configuration is disclosed in which a plurality of switches are displayed on a monitor, the switch is remotely manipulated and selected by input of voice or a visual line of an operator of the endoscope, and decision and execution are made by a foot switch. In addition. JP2001-299691A discloses a configuration in which voice input is enabled by manipulating a foot switch in order to prevent an operation error due to vocalization by a person other than an operator in the configuration of JP1999-332883A (JP-H11-332883A).

### SUMMARY OF THE INVENTION

Generally, an endoscope comprises an insertion part to be inserted into a living body and an operating part for operating the insertion part, and the operating part is provided on a part of the endoscope gripped by an operator. That is, the operator manipulates the insertion part by the operating part at a hand while gripping the endoscope, and this manipulation requires high concentration. In addition, the operator is required to perform various manipulations with respect to the endoscope apparatus, such as a manipulation of switching a display mode of an image while manipulating the insertion part.

However, in the related art, it is not possible to easily perform a manipulation for causing the endoscope apparatus to execute an operation intended by the operator while manipulating the insertion part of the endoscope at the hand by the operator, and thus operability is deteriorated.

For example, in the configurations of JP1999-332883A (JP-H11-332883A) and JP2001-299691A, it is necessary to manipulate the foot switch in order to execute the operation intended by the operator. For this reason, the operator is forced to perform a highly difficult work of performing a manipulation at foot while concentrating on the manipulation at the hand with respect to the endoscope.

The present invention is made in view of the circumstances described above, and is to provide endoscope apparatus, a control method, a non-transitory computer readable recording medium storing a control program, and an endoscope system which can improve operability.

An aspect of the present invention relates to an endoscope apparatus that is connected to an endoscope having an insertion part to be inserted into a living body and an operating part, and displays an image associated with an operation capable of being executed by the endoscope apparatus on a part of a display screen, the apparatus comprising a detection unit that detects a visual line position of an operator of the endoscope, and a control unit that determines whether or not the operator gazes at the image based on the visual line position detected by the detection unit, and executes the operation associated with the image based on a result of the determination and a command signal for giving a command for operation execution, the command signal being input in response to a manipulation by the operator with respect to the operating part.

Another aspect of the present invention relates to a control method of an endoscope apparatus that is connected to an endoscope having an insertion part to be inserted into a living body and an operating part, the method comprising displaying an image associated with an operation capable of being executed by the endoscope apparatus on a part of a display screen, detecting a visual line position of an operator of the endoscope, determining whether or not the operator gazes at the image based on the detected visual line position, and executing the operation associated with the image based on a result of the determination and a command signal for giving a command for operation execution, the command signal being input in response to a manipulation by the operator with respect to the operating part.

Still another aspect of the present invention relates to a non-transitory computer readable recording medium storing a control program of an endoscope apparatus that is connected to an endoscope having an insertion part to be inserted into a living body and an operating part, the program causing a computer to execute a step of displaying an image associated with an operation capable of being executed by the endoscope apparatus on a part of a display screen, a step of detecting a visual line position of an operator of the endoscope, a step of determining whether or not the operator gazes at the image based on the detected visual line position, and a step of executing the operation associated with the image based on a result of the determination and a command signal for giving a command for operation execution, the command signal being input in response to a manipulation by the operator with respect to the operating part.

Still another aspect of the present invention relates to an endoscope system comprising the endoscope apparatus, the endoscope, and the display screen.

According to the present invention, it is possible to provide an endoscope apparatus, a control method, a non-transitory computer readable recording medium storing a control program, and an endoscope system which can improve operability.

### BRIEF DESCRIPTION OF THE DRAWINGS

FIG. 1 is a block diagram showing an example of an endoscope system 100 comprising an endoscope apparatus 120, which is an embodiment of an endoscope apparatus of the present invention.

FIG. 2 is a diagram showing an example of a schematic configuration of an endoscope 110.

FIG. 3 is a diagram showing an example of a display device 130 and a button.

FIG. 4 is a diagram showing a specific example of an operation control by the endoscope apparatus 120.

FIG. 5 is a flowchart showing an example of an operation control process by the endoscope apparatus 120.

FIG. 6 is a diagram showing an example of display of an auxiliary image 620 in a case in which a visual line position is outside a screen 321.

FIG. 7 is a diagram showing another example of the display device 130 and the button.

FIG. 8 is a diagram showing an example of display of the auxiliary image 620 in a case in which the visual line position is outside the screen 321 in the example shown in FIG. 7.

FIG. 9 is a diagram showing another example of the endoscope system 100.

### DESCRIPTION OF THE PREFERRED EMBODIMENTS

Hereinafter, an embodiment of the present invention will be described with reference to the drawings.

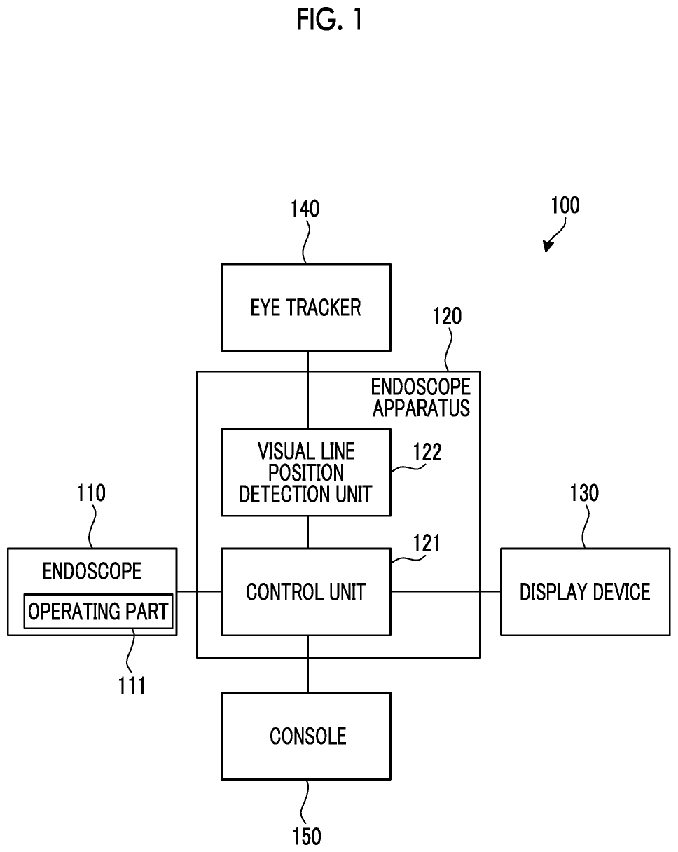

FIG. 1 is a block diagram showing an example of an endoscope system 100 comprising an endoscope apparatus 120, which is the embodiment of an endoscope apparatus of the present invention. The endoscope system 100 comprises an endoscope 110, an endoscope apparatus 120, a display device 130, and an eye tracker 140. In addition, the endoscope system 100 may further comprise a console 150.

The endoscope 110 comprises at least one of an imaging element that obtains an endoscope image in a living body or an ultrasound oscillator that obtains an ultrasound image in the living body. Although not shown, here, a configuration example will be described in which the endoscope 110 comprises both the imaging element and the ultrasound oscillator.

The imaging element images image light in the living body and outputs an imaging signal. For example, the imaging element is configured by a solid-state imaging element, such as a complementary metal-oxide-semiconductor (CMOS) type image sensor or a charge-coupled device (CCD) type image sensor.

For example, the endoscope 110 is provided with an illumination window (not shown) that irradiates an inner wall of a body cavity in the living body with light emitted from a light source (not shown) provided in the endoscope apparatus 120, and the imaging element performs imaging by reflected light of the light.

The ultrasound oscillator is an oscillator that oscillates an ultrasound wave and emits the oscillated ultrasound wave. In addition, the ultrasound oscillator also acts as an ultrasound transducer that receives an echo signal of the emitted ultrasound wave and outputs the received echo signal.

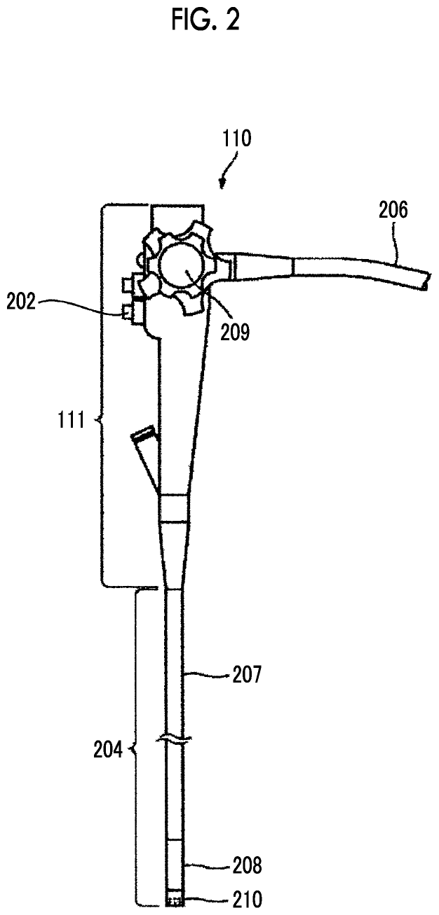

In addition, the endoscope 110 comprises an insertion part (see FIG. 2) to be inserted into the living body and an operating part 111. The operating part 11 is provided at a part of the endoscope that is gripped by an operator (for example, a doctor) of the endoscope (for example, a base end part of the insertion part). A configuration of the endoscope 110 will be described below in FIG. 2.

The endoscope apparatus 120 is connected to the endoscope 110 and performs a control of displaying an image based on the signal obtained by the endoscope 110 on the display device 130. Specifically, the endoscope apparatus 120 comprises a processor for the endoscope image (not shown) that controls drive of the imaging element of the endoscope 110, performs various pieces of image processing on the imaging signal output from the imaging element of the endoscope 110, and generates the endoscope image. In addition, the endoscope apparatus 120 comprises a processor for the ultrasound image (not shown) that controls drive of the ultrasound oscillator of the endoscope 110, performs various pieces of image processing on the echo signal output from the ultrasound oscillator of the endoscope 110, and generates the ultrasound image.

Moreover, the endoscope apparatus 120 generates an image for observation to be displayed by the display device 130, the image for observation including at least one of the endoscope image obtained by the endoscope image processor or the ultrasound image obtained by the ultrasound image processor. In addition, the endoscope apparatus 120 includes a digital scan converter (DSC) (not shown) that converts the generated image for observation into an image signal in accordance with a scanning method of the display device 130 (raster conversion), and performs various pieces of image processing, such as gradation processing, on the converted image signal. Moreover, the endoscope apparatus 120 controls the display of the image for observation by the display device 130 by outputting the image for observation, which is subjected to the image processing by the DSC, to the display device 130.

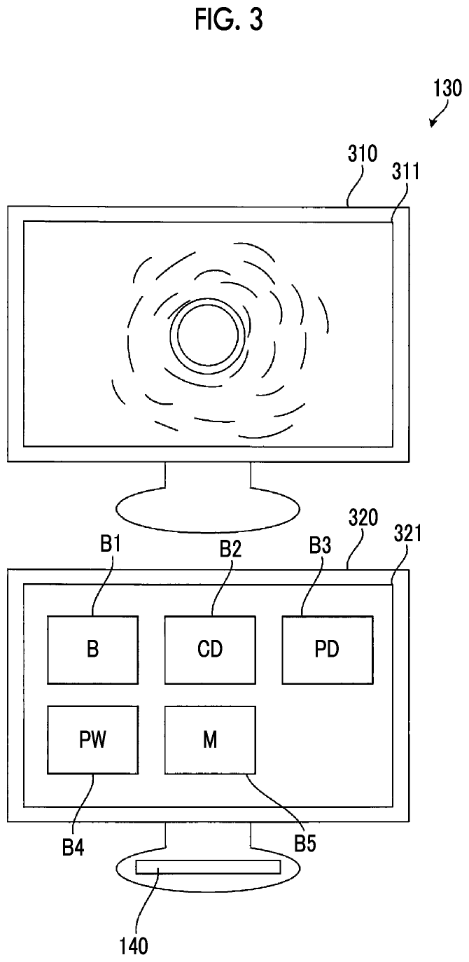

Further, the endoscope apparatus 120 can execute various operations (specific examples thereof will be described below in FIG. 3 and the like), and comprises a control unit 121 and a visual line position detection unit 122 as a configuration for executing an operation desired by the operator among these operations. The visual line position detection unit 122 configures a detection unit.

The control unit 121 displays the button associated with the operation capable of being executed by the endoscope apparatus 120 (self-apparatus) on a part of a display screen of the display device 130. This button is an image (icon) for the operator to select the operation to be executed from among the operations of the endoscope apparatus 120, and configures the image associated with the operation.

In addition, regarding each of a plurality of the operations of the endoscope apparatus 120, the control unit 121 may display the button associated with the operation. Specific examples of the button displayed by the control of the control unit 121 will be described below with reference to FIG. 3 and the like.

The visual line position detection unit 122 detects the visual line position of the operator of the endoscope 110. The visual line position of the operator is, for example, a position that intersects the visual line of the operator on a surface parallel to the display screen of the display device 130. In the configuration example shown in FIG. 1, the visual line position detection unit 122 detects the visual line position of the operator based on information obtained by the eye tracker 140 that detects the visual line position of the operator and the like.

As the eye tracker 140, an eye tracker of various methods, such as a method of detecting the visual line position from an image obtained by imaging a face of the operator with the imaging apparatus, a method of detecting the visual line position by using a special contact lens attached to eyes of the operator, and a method of measuring a potential generated by muscles and the like that move eyeballs of the operator, can be used.

Here, an example will be described in which the eye tracker of the method of detecting the visual line position from the image obtained by imaging the face of the operator with the imaging apparatus is used as the eye tracker 140. In this case, the eye tracker 140 is configured by, for example, a light source that irradiates the face of the operator with near infrared rays, an imaging apparatus that images the face of the operator irradiated with the near infrared rays by the light source, and a processing circuit that specifies the visual line position of the operator by the processing based on the image of the face obtained by the imaging apparatus.

Note that the eye tracker 140 may be configured by being connected to the endoscope apparatus 120 as in the configuration example shown in FIG. 1, or may be configured by being incorporated into the endoscope apparatus 120 as the visual line position detection unit 122 (not shown).

The control unit 121 determines whether or not the operator gazes at the button displayed on the display screen of the display device 130 based on the visual line position of the operator detected by the visual line position detection unit 122. A state in which the operator gazes at the button means a state in which the visual line of the operator is directed toward the button, that is, the operator concentrates his/her eyesight on the button.

For example, the control unit 121 may determine whether or not the operator gazes at the button by determining whether or not the visual line position is included in a region in which the button is displayed in the display screen of the display device 130. Note that a method of determining whether or not the operator gazes at the button is not limited to this.

For example, the control unit 121 may determine whether or not the operator gazes at the button by determining whether or not a distance between a center position of the region in which the button is displayed in the display screen of the display device 130 and the visual line position is a threshold value or more.

In addition, a command signal (trigger signal) for giving a command for operation execution by the endoscope apparatus 120, which is output in response to the manipulation from the operator with respect to the operating part 111 of the endoscope 110, is input to the control unit 121. Moreover, the control unit 121 performs a control of executing the operation associated with the button based on the determination as to whether or not the operator gazes at the button and the input of the command signal described above.

Specifically, in a case in which the command signal is input in a state in which it is determined that the operator gazes at the button, the control unit 121 executes the operation associated with the button.

The display device 130 displays the image for observation described above, the image, such as a button, or the like by the control from the control unit 121. For example, the display device 130 is configured by being integrally provided with the endoscope apparatus 120. Alternatively, a configuration may be adopted in which the display device 130 is provided outside the endoscope apparatus 120 and controlled by the endoscope apparatus 120 by communicating with the endoscope apparatus 120. In this case, the communication between the display device 130 and the endoscope apparatus 120 may be wired communication or wireless communication. In addition, the display device 130 may include a plurality of display devices.

The console 150 is a user interface for the operator to perform various operations with respect to the endoscope apparatus 120. For example, as the console 150, various user interfaces, such as press buttons, change switches, touch panels, and voice input devices, can be used. The control unit 121 may be able to execute the operation as commanded by the console 150 in addition to the execution of the operation based on the determination and the command signal described above.

Note that the endoscope apparatus 120 includes various processors that collectively control the entire endoscope system 100 and execute a program including a control program to perform processing, a random access memory (RAM), and a read only memory (ROM).

Examples of the various processors include a central processing unit (CPU), which is a general-purpose processor that executes a program and performs various pieces of processing, programmable logic device (PLD), which is a processor of which a circuit configuration can be changed after manufacturing, such as field programmable gate array (FPGA), or a dedicated electric circuit, which is a processor having a circuit configuration specially designed for executing specific processing, such as an application specific integrated circuit (ASIC), and the like.

The structure of these various processors is, more specifically, an electric circuit in which circuit elements, such as semiconductor elements, are combined. The endoscope apparatus 120 may be configured by one of the various processors, or may be configured by a combination of two or more processors of the same type or different types (for example, a combination of a plurality of the FPGAs or a combination of the CPU and the FPGA).

FIG. 2 is a diagram showing an example of a schematic configuration of the endoscope 110. The endoscope 110 shown in FIG. 2 mainly comprises the operating part 111, an insertion part 204, and a universal cord 206 connected to the control unit 121 via a connector unit (not shown) of the endoscope apparatus 120.

The insertion part 204 is mostly a flexible tube 207 that bends in any direction along an insertion path. A bendable part 208 is connected to a distal end of the flexible tube 207, and a distal end part 210 is connected to the distal end of the bendable part 208. The bendable part 208 is provided so that the distal end part 210 is directed in a desired direction, and a bending operation can be performed by moving rotationally a bending operation knob 209 provided on the operating part 111. Although not shown, the distal end part 210 is provided with the imaging element, the ultrasound oscillator, the illumination window, and the like described above.

The operating part 111 is a part to be gripped by the operator, and is provided at the base end of the flexible tube 207 (end part of the flexible tube 207 opposite to the distal end part 210), and comprises each component that receives the manipulation from the operator. For example, the operating part 11 is provided with a push button 202 and the like in addition to the bending operation knob 209 described above. As a trigger switch for outputting the command signal described above to the control unit 121, for example, the push button 202 can be used. In this case, in a case in which the operator presses the push button 202, the command signal described above is input to the control unit 121 via the universal cord 206.

In this case, the operator can cause the endoscope apparatus 120 to execute the operation corresponding to the button by pressing the push button 202 in a state in which the operator meets his/her visual line with the button displayed in the display device 130 while manipulating the distal end part 210 by using the bending operation knob 209 in a state of gripping the operating part 111.

As a result, both the manipulation of the distal end part 210 and the manipulation of the endoscope apparatus 120 can be performed by the operating part 111 at a hand of the operator.

Therefore, for example, the work of the operator can be facilitated as compared with a configuration in which the manipulation of the distal end part 210 and the manipulation of the endoscope apparatus 120 are performed by using the operating part 111 at the hand of the operator and a foot switch at the foot of the operator, respectively. Note that the manipulation of the distal end part 210 includes a manipulation of maintaining a position or a posture of the distal end part 210.

Note that the trigger switch for outputting the command signal to the control unit 121 is not limited to the push button 202, but may be another push button provided on the operating part 111, or may be a touch sensor and the like provided on the operating part 111 other than the push button.

FIG. 3 is a diagram showing an example of the display device 130 and the button. In the example shown in FIG. 3, the display device 130 is configured by two monitors 310 and 320. The monitor 310 has a screen 311 that displays the image for observation, such as the endoscope image or the ultrasound image obtained by the endoscope 110, by the control from the endoscope apparatus 120. In the example shown in FIG. 3, the ultrasound image is displayed on the monitor 310 as the image for observation.

The monitor 320 has a screen 321 that displays the buttons associated with the plurality of operations capable of being executed by the endoscope apparatus 120 by the control from the endoscope apparatus 120. The screen 321 configures the display screen.

In the example shown in FIG. 3, the monitor 320 displays buttons B1 to B5. The buttons B1 to B5 are associated with a shift to a brightness (B) mode, a shift to a color Doppler (CD) mode, a shift to a power Doppler (PD) mode, a shift to a pulse wave (PW) mode, and a shift to a motion (M) mode, in advance.

The B mode, the CD mode, the PD mode, the PW mode, and the M mode are ultrasound image generation modes different from each other. For example, the B mode is a mode in which amplitude of an ultrasound echo is converted into brightness and a tomographic image is displayed.

The CD mode is a mode in which information on a blood flow velocity including a direction obtained by a Doppler method is superimposed and displayed on a B mode image in color. The PW mode is a mode in which a velocity (for example, the blood flow velocity) of an ultrasound echo source detected based on the transmission and reception of a pulse wave is displayed.

The PD mode is a mode in which power information of a Doppler signal in a color Doppler method is superimposed and displayed on the B mode image in color. The M mode is a mode in which a temporal change when paying attention to a certain straight line on the tomographic image is imaged and displayed.

The buttons shown in FIG. 3 are examples, and the display of a part of the buttons shown in FIG. 3 may be omitted, or the button associated with the shift to another mode may be further displayed on the screen 321. Examples of the other mode include an amplitude (A) mode.

In addition, the operation associated with the button displayed on the screen 321 is not limited to the shift to a specific ultrasound image generation mode. For example, the operation associated with the button displayed on the screen 321 may be switching of the image displayed on the screen 311 among the ultrasound image and the endoscope image, storage of the image for observation displayed on the screen 311 as a motion picture or a still picture, or switching a diagnosis support function, such as measurement or the button displayed on the screen 321.

In addition, in the configuration example shown in FIG. 3, an imaging apparatus of the eye tracker 140 is provided below the monitor 320. In a case in which the operator selects the buttons B1 to B5 displayed on the monitor 320, the face of the operator is directed to the monitor 320. Therefore, by providing the imaging apparatus of the eye tracker 140 on the monitor 320, the visual line position of the operator can be detected with high accuracy.

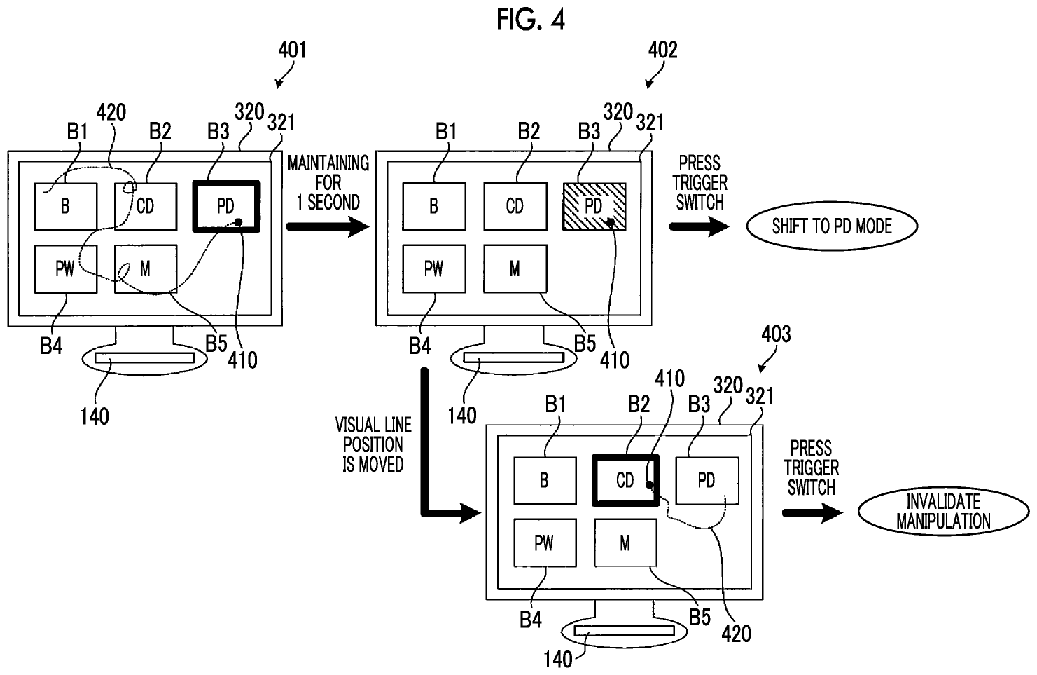

FIG. 4 is a diagram showing a specific example of the operation control by the endoscope apparatus 120. A state 401 is a state in which the visual line position of the operator moves within the screen 321. A cursor 410 is an image showing the visual line position of the operator, and is displayed at a current visual line position of the operator on the screen 321 detected by the visual line position detection unit 122. In the example shown in FIG. 4, the cursor 410 is configured by a circular image, but the image that configures the cursor 410 is not limited to this. A visual line trajectory 420 is a trajectory of the movement of the cursor 410.

The control unit 121 sequentially updates the cursor 410 and the visual line trajectory 420 based on a result of the detection by the visual line position detection unit 122. Note that the control unit 121 does not have to display the visual line trajectory 420 on the screen 321.

In addition, the control unit 121 may perform a control of highlighting the button in a case in which the cursor 410 is positioned in any region of the buttons B1 to B5. Highlighting of the button means making the button stand out more than the other buttons.

For example, in the state 401 of FIG. 3, since the cursor 410 is positioned in the region of the button B3, the control unit 121 highlights the button B3 by making an outer line of the button B3 thicker than the outer line of the other buttons. As a result, the operator can easily grasp that the button as commanded by his/her visual line is the button B3. Note that the highlighting of the button is not limited to this, and various highlighting, such as changing a color of the button or changing a size of the button, can be used.

In addition, in the state 401, the control unit 121 sets the buttons B1 to B5 in an inactive state. Here, an active state of the button is a state in which the control unit 121 executes the operation corresponding to the button in a case in which the command signal described above is input when the cursor 410 is positioned in the region of the button.

On the other hand, the inactive state of the button is a state in which the control unit 121 does not execute the operation corresponding to the button even in a case in which the command signal described above is input when the cursor 410 is positioned in the region of the button. That is, in the state 401, since the button B3 is set to the inactive state, even in a case in which the trigger switch (for example, the push button 202) is pressed and the command signal is input, the operation corresponding to the button B3 is not executed.

In the state 401, in a case in which the state in which the cursor 410 is positioned in the region of the button B3 is maintained for 1 second or longer, a state 402 is obtained. In the state 402, the control unit 121 sets the button B3 to the active state. At this time, the control unit 121 may notify the operator that the button B3 is placed in the active state by changing the display aspect of the button B3.

In the example shown in FIG. 4, the control unit 121 notifies the operator that the button B3 is placed in the active state by making the color of the button B3 different from that of the other buttons. Note that the display aspect of the button in the active state is not limited to this, and various display aspects, such as changing the color different from that of the button in the inactive state and changing the size larger than that of the button in the inactive state, can be used.

In a case in which the trigger switch is pressed in the state 402, the control unit 121 executes the shift to the PD mode, which is the operation corresponding to the button B3. As a result, the ultrasound image displayed on the monitor 310 is switched to the ultrasound image in the PD mode.

On the other hand, in the state 402, in a case in which the cursor 410 moves out of the region of the button B3 and moves to the region of the button B2, a state 403 is obtained. In the state 403, the control unit 121 restores the button B3 to the inactive state. In addition, the control unit 121 highlights the button B2. Note that at this point in time, a state in which the cursor 410 is positioned in the region of the button B2 continues for shorter than 1 second, and the button B2 remains in the inactive state.

In a case in which the trigger switch is pressed in the state 403, the button B2 is placed in the inactive state, and thus the control unit 121 does not execute the operation corresponding to the button B2, and invalidates the operation. As a result, for example, in a case in which the visual line of the operator swings or the visual line position detection unit 122 erroneously detects the visual line immediately before the trigger switch is pressed, it is possible to suppress the execution of the operation not intended by the operator.

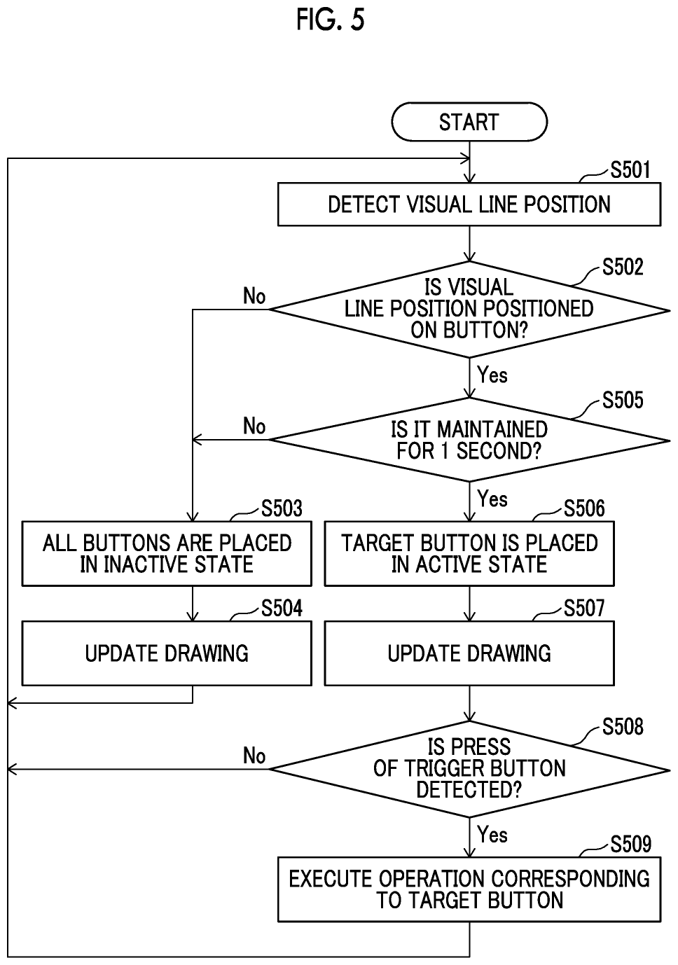

FIG. 5 is a flowchart showing an example of an operation control process by the endoscope apparatus 120. First, the endoscope apparatus 120 detects the visual line position of the operator (step S501). Then, the endoscope apparatus 120 determines whether or not the visual line position detected in step S501 is positioned on the button displayed on the screen 321 (step S502). For example, in the example shown in FIG. 3, the endoscope apparatus 120 determines whether or not the visual line position is positioned in any region of the buttons B1 to B5.

In step S502, in a case in which the visual line position is not positioned on the button (step S502: No), the endoscope apparatus 120 sets all the buttons to the inactive state (step S503). Then, the endoscope apparatus 120 updates the drawing on the screen 321 (step S504), and returns to step S501. Examples of the update of the drawing in step S504 include the movement of the cursor 410, and switching of highlighting of each button.

In step S502, in a case in which the visual line position is positioned on the button (step S502: Yes), the endoscope apparatus 120 determines whether or not a state in which the detected visual line position is positioned on the button (hereinafter, referred to as a target button) is maintained for 1 second or longer (step S505). For example, the endoscope apparatus 120 stores a history of results of the determination in step S502 in a memory, such as the RAM, and makes the determination in step S505 based on this history.

In step S505, in a case in which the state in which the visual line position is positioned on the target button is not maintained for 1 second or longer (step S505: No), the endoscope apparatus 120 shifts to step S503. In a case in which the state in which the visual line position is positioned on the target button is maintained for 1 second or longer (step S505: Yes), the endoscope apparatus 120 sets the target button to the active state (step S506).

Then, the endoscope apparatus 120 updates the drawing on the screen 321 (step S507). Examples of the update of the drawing in step S507 include the movement of the cursor 410 and the display that the target button is placed in the active state (for example, changing the color of the target button).

Then, the endoscope apparatus 120 determines whether or not the press of a trigger button (for example, the push button 202) is detected (step S508). That is, the endoscope apparatus 120 determines whether or not the command signal described above is input.

In a case in which the press of the trigger button is not detected in step S508 (step S508: No), the endoscope apparatus 120 returns to step S501. In a case in which the press of the trigger button is detected (step S508: Yes), the endoscope apparatus 120 executes the operation corresponding to the target button (step S509), and returns to step S501.

Step S501 shown in FIG. 5 is executed by the visual line position detection unit 122 of the endoscope apparatus 120, for example. Steps S502 to S509 shown in FIG. 5 are executed by the control unit 121 of the endoscope apparatus 120, for example.

As described with reference to FIGS. 4 and 5, the endoscope apparatus 120 executes the operation corresponding to the button in a case in which the command signal is input in a state in which it is determined that the operator gazes at the button continuously for a predetermined time (for example, 1 second) or longer. As a result, it is possible to suppress the execution of the operation not intended by the operator.

In addition, the endoscope apparatus 120 changes the display aspect of the button in a case in which a state in which it is determined that the operator gazes at the button continues for the time described above, that is, the button is placed in the active state. As a result, the operator can easily grasp that the operation corresponding to the button at which the operator gazes is executed by pressing the trigger switch.

A case has been described in which 1 second is used as the time until the button is placed in the active state (predetermined time), but the time until the button is placed in the active state may be shorter than 1 second or longer than 1 second. In addition, the time until the button is placed in the active state may be set to 0 seconds, that is, the button may be immediately in the active state in a case in which the operator gazes at the button.

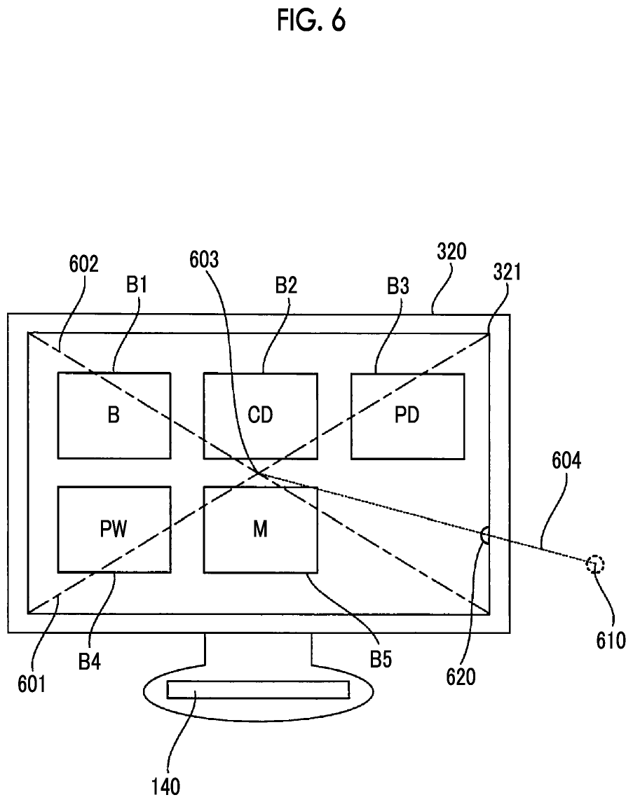

FIG. 6 is a diagram showing an example of the display of an auxiliary image 620 in a case in which the visual line position is outside the screen 321. A visual line position 610 shown in FIG. 6 is the visual line position detected by the visual line position detection unit 122. As shown in FIG. 6, in a case in which the visual line position 610 detected by the visual line position detection unit 122 is outside the screen 321, the control unit 121 may display the auxiliary image 620 on the screen 321 instead of the cursor 410.

Virtual diagonal lines 601 and 602 of FIG. 6 virtually show two diagonal lines of the rectangular screen 321. A center 603 is an intersection of the virtual diagonal lines 601 and 602, that is, the center of the screen 321. A virtual line segment 604 of FIG. 6 virtually shows a line segment connecting the center 603 and the visual line position 610. The virtual diagonal lines 601 and 602, the center 603, and the virtual line segment 604 do not have to be actually displayed on the screen 321.

The control unit 121 displays the auxiliary image 620 at a position of an end part of the screen 321 that intersects with the virtual line segment 604. As a result, the auxiliary image 620 showing the direction of the visual line position 610 can be displayed at the end part of the screen 321 between the center 603 of the screen 321 and the visual line position 610 among the end parts of the screen 321.

In the example shown in FIG. 6, the auxiliary image 620 is a semi-circular image which is a part included in the screen 321 in a circle centered on a position intersecting the virtual line segment 604 in the end part of the screen 321. In addition, the auxiliary image 620 is the image having the color different from that of the cursor 410. In this way, by using the image different from the cursor 410 as the auxiliary image 620, the operator can easily grasp that the visual line position 610 is not positioned at the position of the auxiliary image 620 and the visual line position 610 is positioned in the direction shown by the auxiliary image 620.

In addition, the control unit 121 sequentially updates the auxiliary image 620 based on the result of the detection by the visual line position detection unit 122. Moreover, in a case in which the visual line position 610 is inside the screen 321, the control unit 121 displays the cursor 410 at the visual line position 610 instead of the auxiliary image 620.

As described above, in a state in which the visual line position 610 is detected inside the screen 321, the endoscope apparatus 120 displays the cursor 410 indicating the visual line position 610 at the position at which the visual line position 610 is detected on the screen 321. On the other hand, in a state in which the visual line position 610 is detected outside the screen 321, the endoscope apparatus 120 displays the auxiliary image 620 showing the direction of the visual line position 610 at the end part between the center 603 of the screen 321 and the visual line position 610 among the end parts of the screen 321.

As a result, even in a case in which the visual line position 610 detected by the visual line position detection unit 122 deviates from the actual visual line position of the operator due to a tracking failure of the eye tracker 140 and the like, the operator knows the direction of the detected his/her visual line position 610, and thus can intuitively grasp a direction of moving the visual line from the current state for the cursor 410 to fit the desired button. Therefore, it is possible to suppress the confusion of the manipulation based on the tracking failure of the eye tracker 140 and the like.

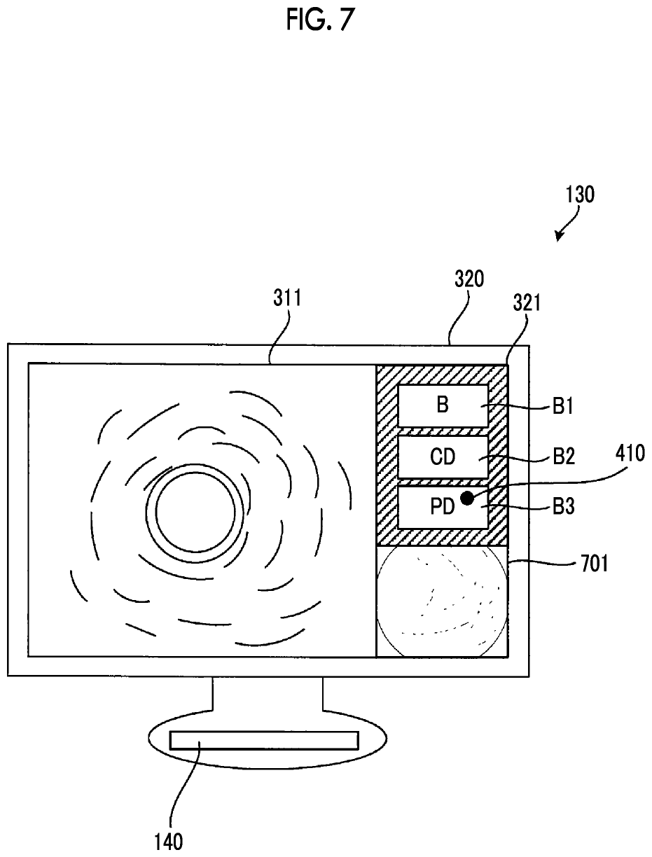

FIG. 7 is a diagram showing another example of the display device 130 and the button. In FIG. 7, the same parts as those shown in FIG. 3 are given by the same reference numerals, and the description thereof will be omitted. In the example shown in FIG. 7, the display device 130 is configured by one monitor 320. By software processing, the screen of the monitor 320 is divided into three screens 311, 321 and 701.

The monitor 320 displays the ultrasound image obtained by the endoscope 110 on the screen 311 by the control from the endoscope apparatus 120. In addition, the monitor 320 displays the buttons associated with the plurality of operations capable being executed by the endoscope apparatus 120 on the screen 321 (diagonal line part in FIG. 7) by the control from the endoscope apparatus 120. In addition, the monitor 320 displays the endoscope image obtained by the endoscope 110 on the screen 701 by the control from the endoscope apparatus 120.

In the example shown in FIG. 7, the monitor 320 displays the buttons B1 to B3 on the screen 321.

The operator gazes at any of the buttons B1 to B3 displayed on the screen 321 for 1 second or longer, and presses the push button 202 of the endoscope 110 in that state, so that the ultrasound image generation mode corresponding to the gazed button among the buttons B1 to B3 can be activated.

For example, in a case in which the operator gazes at the button B3 for 1 second or longer and presses the push button 202 of the endoscope 110 in that state, the ultrasound image displayed on the screen 311 is switched to the ultrasound image in the PD mode.

As shown in FIG. 7, the endoscope apparatus 120 may display the image for observation obtained by the endoscope 110 on a screen 311 different from the screen 321, which is included in the monitor 320 having the screen 321. As a result, the operator can view the image for observation obtained by the endoscope 110 and the buttons B1 to B3 for causing the endoscope apparatus 120 to execute the desired operation on one monitor 320.

Therefore, the operator can cause the endoscope apparatus 120 to execute the desired operation by moving the visual line with a small amount and pressing the trigger switch while performing the observation with the image for observation. Therefore, it is possible to improve the operability.

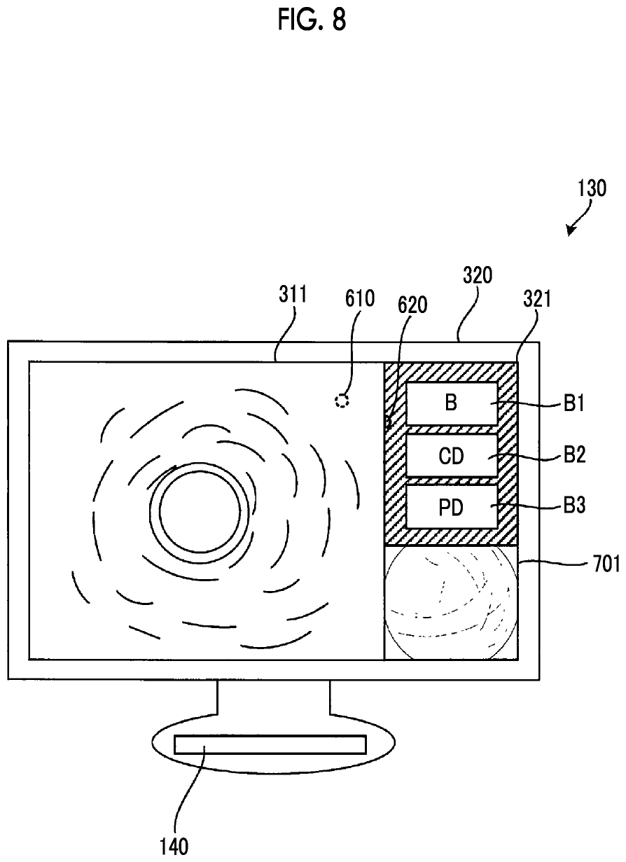

FIG. 8 is a diagram showing an example of the display of the auxiliary image 620 in a case in which the visual line position is outside the screen 321 in the example shown in FIG. 7. In the example shown in FIG. 7, as shown in FIG. 8, in a case in which the visual line position 610 detected by the visual line position detection unit 122 is outside the screen 321, the control unit 121 may display the auxiliary image 620 on the screen 321 instead of the cursor 410.

Specifically, the control unit 121 displays the auxiliary image 620 at the end part between the center of the screen 321 and the visual line position 610 among the end parts of the screen 321, as in the example shown in FIG. 6. That is, the control unit 121 displays the cursor 410 or the auxiliary image 620 within a range of the screen 321 and does not display the cursor 410 and the auxiliary image 620 on the screen 311.

As a result, the cursor 410 or the auxiliary image 620 are not displayed on the screen 311 that displays the image for observation, such as the ultrasound image, so that it is possible to suppress interfering of the cursor 410 or the auxiliary image 620 with the diagnosis by the image for observation.

(Modification Example of Display Device 130)

Although the example has been described in which the display device 130 is configured by the free-supporting monitors 310 and 320 with reference to FIGS. 3, 7, and 8, the configuration of the display device 130 is not limited to this. For example, the display device 130 may be configured by a display device, such as a touch panel, included in the console 150, a projector, or the like.

(Extraction of Facial Region of Operator)

In the configuration in which the eye tracker 140 is included in the visual line position detection unit 122, the eye tracker 140 may store facial feature information of the operator in advance. In this case, the eye tracker 140 extracts the facial region of the operator from the captured image obtained by the imaging apparatus that images the operator based on the facial feature information of the operator. Various methods used in face authentication and the like can be used to extract the facial region.

Moreover, the eye tracker 140 detects the visual line position of the operator based on the image of the extracted facial region. As a result, for example, even in a case in which a person other than the operator (for example, an assistant of the operator or a patient) is reflected in the captured image, the visual line position of the operator can be detected with high accuracy.

Alternatively, a marker having an optical feature may be attached to the operator, and the facial region of the operator may be extracted by using this marker. The optical feature is a feature, such as specific shape or color, which can be extracted by the image processing, such as image matching. As an example, a star-shaped seal is attached to a chest of the operator or the like as the marker.

The eye tracker 140 detects a position of the marker in the captured image obtained by the imaging apparatus that images the operator attached with the marker described above, and extracts the facial region of the operator in the captured image based on the detected position of the marker. For example, in a case in which the marker is attached to the chest of the operator, the position of the detected marker is the position of the chest of the operator, so that it can be determined that the region above the position is the facial region of the operator.

In this case as well, the eye tracker 140 detects the visual line position of the operator based on the image of the extracted facial region. As a result, for example, even in a case in which a person other than the operator is reflected in the captured image, the visual line position of the operator can be detected with high accuracy.

As described above, the eye tracker 140 may limit the region for detecting the visual line position to the facial region of the operator in the captured image obtained by the imaging apparatus by using the face identification or the marker. As a result, the visual line position of the operator can be detected with high accuracy.

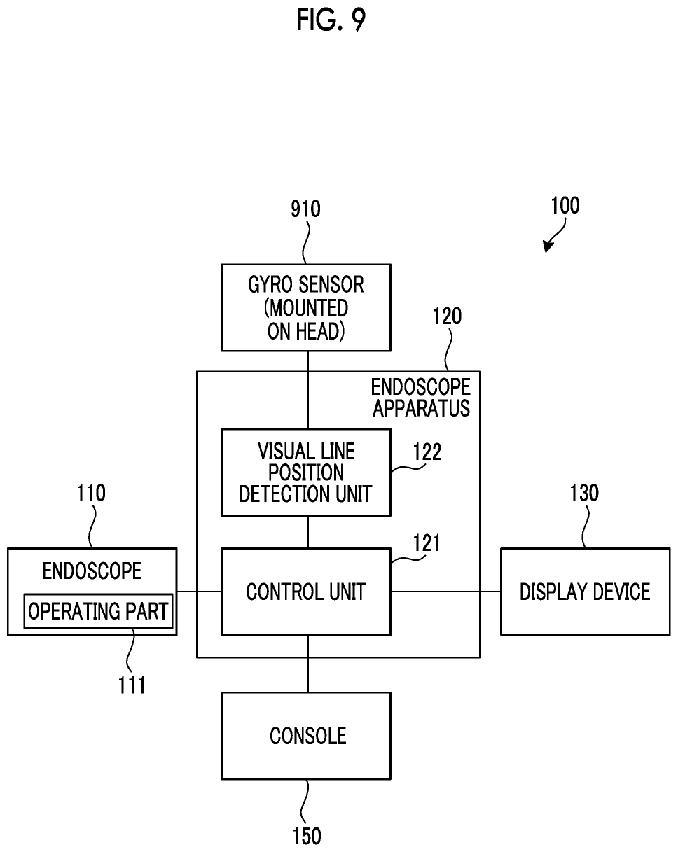

FIG. 9 is a diagram showing another example of the endoscope system 100. In FIG. 9, the same parts as those shown in FIG. 1 are given by the same reference numerals, and the description thereof will be omitted. The endoscope system 100 may include a gyro sensor 910 instead of the eye tracker 140 as shown in FIG. 9. The gyro sensor 910 is mounted to the head of the operator, and outputs the information obtained by measuring an angular velocity of the head of the operator and indicating a three-dimensional posture change of the head of the operator to the endoscope apparatus 120.

The visual line position detection unit 122 of the endoscope apparatus 120 detects the visual line position of the operator based on the information output from the gyro sensor 910. That is, since the movement of the visual line of the operator is linked to the movement of the head of the operator to some extent, the visual line position of the operator can be indirectly detected based on the information output from the gyro sensor 910.

As described above, the visual line position detection by the visual line position detection unit 122 is not limited to the information obtained by the eye tracker 140, but the detection can be performed based on the information obtained by another unit, such as the gyro sensor 910.

In the endoscope apparatus 120 configured as described above, the image associated with the operation capable of being executed is displayed on a part of the display screen, and it is determined whether or not the operator gazes at the image based on the visual line position of the operator of the endoscope. Moreover, the endoscope apparatus 120 executes the operation associated with the image based on the result of the determination and the command signal input in response to the manipulation with respect to the operating part of the endoscope.

As a result, both the manipulation of the endoscope and the manipulation of the endoscope apparatus 120 can be performed by the operating part at the hand of the operator.

Therefore, for example, the work of the operator can be facilitated as compared with a configuration in which the manipulation of the endoscope and the manipulation of the endoscope apparatus 120 are performed by using the operating part at the hand of the operator and the foot switch at the foot of the operator, respectively. Therefore, it is possible to improve the operability.

In addition, by using the eye tracker or the gyro sensor, the operability can be improved without using a large-scale device, such as a head-mounted display.

The control program, which is stored in the ROM or the like of the endoscope apparatus 120, is stored in a computer-readable non-transitory storage medium. Examples of such a “computer-readable storage medium” include an optical medium, such as a compact disc-rom (CD-ROM), and a magnetic storage medium, such as a universal serial bus (USB) memory, or a memory card. In addition, such a program can also be provided by downloading via a network, such as the Internet.

As described above, at least the following matters are described in the present specification.

(1)

An endoscope apparatus that is connected to an endoscope having an insertion part to be inserted into a living body and an operating part, and displays an image associated with an operation capable of being executed by the endoscope apparatus on a part of a display screen, the apparatus comprising a detection unit that detects a visual line position of an operator of the endoscope, and a control unit that determines whether or not the operator gazes at the image based on the visual line position detected by the detection unit, and executes the operation associated with the image based on a result of the determination and a command signal for giving a command for operation execution, the command signal being input in response to a manipulation by the operator with respect to the operating part.

(2)

The endoscope apparatus according to (1), in which the control unit executes the operation associated with the image in a case in which the command signal is input in a state in which it is determined that the operator gazes at the image.

(3)

The endoscope apparatus according to (2), in which the control unit executes the operation associated with the image in a case in which the command signal is input in a state in which it is determined that the operator gazes at the image continuously for a time equal to or longer than a predetermined time.

(4)

The endoscope apparatus according to (3), in which the control unit changes a display aspect of the image in a case in which a state in which it is determined that the operator gazes at the image continues for the time.

(5)

The endoscope apparatus according to any one of (1) to (4), in which the insertion part has a flexible tube, and the operating part is provided at a base end of the flexible tube.

(6)

The endoscope apparatus according to any one of (1) to (5), in which the operating part is an operating part used in a state of being gripped by the operator.

(7)

The endoscope apparatus according to any one of (1) to (6), in which the control unit displays a cursor indicating the visual line position at a position on the display screen at which the visual line position is detected in a state in which the visual line position is detected inside the display screen, and displays an auxiliary image indicating a direction of the visual line position at an end part between a center of the display screen and the visual line position among end parts of the display screen in a state in which the visual line position is detected outside the display screen.

(8)

The endoscope apparatus according to any one of (1) to (7), in which the detection unit extracts a facial region of the operator from a captured image obtained by an imaging apparatus that images the operator based on facial feature information of the operator, and detects the visual line position based on an image of the extracted facial region.

(9)

The endoscope apparatus according to any one of (1) to (7), in which the detection unit detects a position of a marker having an optical feature in a captured image obtained by an imaging apparatus that images the operator attached with the marker, extracts a facial region of the operator in the captured image based on the detected position of the marker, and detects the visual line position based on an image of the extracted facial region.

(10)

The endoscope apparatus according to any one of (1) to (9), in which the control unit displays an image for observation obtained by the endoscope on a screen, which is included in a monitor having the display screen and different from the display screen.

(11)

The endoscope apparatus according to any one of (1) to (10), in which the detection unit detects the visual line position based on information obtained by an eye tracker.

(12)

The endoscope apparatus according to any one of (1) to (10), in which the detection unit detects the visual line position based on information obtained by a gyro sensor mounted on a head of the operator.

(13)

A control method of an endoscope apparatus that is connected to an endoscope having an insertion part to be inserted into a living body and an operating part, the method comprising displaying an image associated with an operation capable of being executed by the endoscope apparatus on a part of a display screen, detecting a visual line position of an operator of the endoscope, determining whether or not the operator gazes at the image based on the detected visual line position, and executing the operation associated with the image based on a result of the determination and a command signal for giving a command for operation execution, the command signal being input in response to a manipulation by the operator with respect to the operating part.

(14)

A control program of an endoscope apparatus that is connected to an endoscope having an insertion part to be inserted into a living body and an operating part, the program causing a computer to execute a step of displaying an image associated with an operation capable of being executed by the endoscope apparatus on a part of a display screen, a step of detecting a visual line position of an operator of the endoscope, a step of determining whether or not the operator gazes at the image based on the detected visual line position, and a step of executing the operation associated with the image based on a result of the determination and a command signal for giving a command for operation execution, the command signal being input in response to a manipulation by the operator with respect to the operating part.

(15)

An endoscope system comprising the endoscope apparatus according to any one of (1) to (12), the endoscope, and the display screen.

From the above description, the endoscope apparatus according to the following supplementary notes 1 to 12 can be grasped.

[Supplementary Note 1]

An endoscope apparatus that is connected to an endoscope having an insertion part to be inserted into a living body and a trigger switch, and displays an image associated with an operation capable of being executed by the endoscope apparatus on a part of a display screen, the apparatus comprising a processor, in which the processor detects a visual line position of an operator of the endoscope, determines whether or not the operator gazes at the image based on the position of the visual line, and executes the operation associated with the image based on a result of the determination and a command signal for giving a command for operation execution, the command signal being input in response to a manipulation by the operator with respect to the trigger switch.

[Supplementary Note 2]

The endoscope apparatus according to Supplementary Note 1, in which the processor executes the operation associated with the image in a case in which the command signal is input in a state in which it is determined that the operator gazes at the image.

[Supplementary Note 3]

The endoscope apparatus according to Supplementary Note 2, in which the processor executes the operation associated with the image in a case in which the command signal is input in a state in which it is determined that the operator gazes at the image continuously for a time equal to or longer than a predetermined time.

[Supplementary Note 4]

The endoscope apparatus according to Supplementary Note 3, in which the processor changes a display aspect of the image in a case in which a state in which it is determined that the operator gazes at the image continues for the time.

[Supplementary Note 5]

The endoscope apparatus according to any one of Supplementary Notes 1 to 4, in which the insertion part has a flexible tube, and the trigger switch is provided at a base end of the flexible tube.

[Supplementary Note 6]

The endoscope apparatus according to any one of Supplementary Notes 1 to 5, in which the trigger switch is a trigger switch used in a state of being gripped by the operator.

[Supplementary Note 7]

The endoscope apparatus according to any one of Supplementary Notes 1 to 6, in which the processor displays a cursor indicating the visual line position at a position on the display screen at which the visual line position is detected in a state in which the visual line position is detected inside the display screen, and displays an auxiliary image indicating a direction of the visual line position at an end part between a center of the display screen and the visual line position among end parts of the display screen in a state in which the visual line position is detected outside the display screen.

[Supplementary Note 8]

The endoscope apparatus according to any one of Supplementary Notes 1 to 7, in which the processor extracts a facial region of the operator from a captured image obtained by an imaging apparatus that images the operator based on facial feature information of the operator, and detects the visual line position based on an image of the extracted facial region.

[Supplementary Note 9]

The endoscope apparatus according to any one of Supplementary Notes 1 to 7, in which the processor detects a position of a marker having an optical feature in a captured image obtained by an imaging apparatus that images the operator attached with the marker, extracts a facial region of the operator in the captured image based on the detected position of the marker, and detects the visual line position based on an image of the extracted facial region.

[Supplementary Note 10]

The endoscope apparatus according to any one of Supplementary Notes 1 to 9, in which the processor displays an image for observation obtained by the endoscope on a screen, which is included in a monitor having the display screen and different from the display screen.

[Supplementary Note 11]

The endoscope apparatus according to any one of Supplementary Notes 1 to 10, in which the processor detects the visual line position based on information obtained by an eye tracker.

[Supplementary Note 12]

The endoscope apparatus according to any one of Supplementary Notes 1 to 10, in which the processor detects the visual line position based on information obtained by a gyro sensor mounted on a head of the operator.

### EXPLANATION OF REFERENCES

100: endoscope system
110: endoscope
111: operating part
120: endoscope apparatus
121: control unit
122: visual line position detection unit
130: display device
140: eye tracker
150: console
202: push button
204: insertion part
206: universal cord
207: flexible tube
208: bendable part
209: bending operation knob
210: distal end part
310, 320: monitor
311, 321, 701: screen
401 to 403: state
410: cursor
420: visual line trajectory
601, 602: virtual diagonal line
603: center
604: virtual line segment
610: visual line position
620: auxiliary image
910: gyro sensor
B1 to B5: button

What is claimed is:

1. An endoscope apparatus that is connected to an endoscope having an insertion part to be inserted into a living body and a trigger switch, and displays an image associated with an operation capable of being executed by the endoscope apparatus on a part of a display screen, the apparatus comprising a processor that is configured to:
detect a visual line position of an operator of the endoscope;
determine whether or not the operator gazes at the image based on the position of the visual line, and execute the operation associated with the image based on a result of the determination and a command signal for giving a command for operation execution, the command signal being input in response to a manipulation by the operator with respect to the trigger switch.

2. The endoscope apparatus according to claim 1
wherein the processor is configured to execute the operation associated with the image in a case in which the command signal is input in a state in which the processor determines that the operator gazes at the image.

3. The endoscope apparatus according to claim 2
wherein the processor is configured to execute the operation associated with the image in a case in which the command signal is input in a state in which the processor determines that the operator gazes at the image continuously for a time equal to or longer than a predetermined time.

4. The endoscope apparatus according to claim 3
wherein the processor is configured to change a display aspect of the image in a case in which a state in which the processor determines that the operator gazes at the image continues for the time.

5. The endoscope apparatus according to claim 1
wherein the insertion part has a flexible tube, and
wherein the trigger switch is provided at a base end of the flexible tube.

6. The endoscope apparatus according to claim 1
wherein the trigger switch is a trigger switch used in a state of being gripped by the operator.

7. The endoscope apparatus according to claim 1
wherein the processor is configured to display a cursor indicating the visual line position at a position on the display screen at which the visual line position is detected in a state in which the visual line position is detected inside the display screen, and display an auxiliary image indicating a direction of the visual line position at an end part between a center of the display screen and the visual line position among end parts of the display screen in a state in which the visual line position is detected outside the display screen.

8. The endoscope apparatus according to claim 1
wherein the processor is configured to extract a facial region of the operator from a captured image obtained by an imaging apparatus that images the operator based on facial feature information of the operator, and detect the visual line position based on an image of the extracted facial region.

9. The endoscope apparatus according to claim 1
wherein the processor is configured to detect a position of a marker having an optical feature in a captured image obtained by an imaging apparatus that images the operator attached with the marker, extract a facial region of the operator in the captured image based on the detected position of the marker, and detect the visual line position based on an image of the extracted facial region.

10. The endoscope apparatus according to claim 1
wherein the processor is configured to display an image for observation obtained by the endoscope on a screen, which is included in a monitor having the display screen and different from the display screen.

11. The endoscope apparatus according to claim 1
wherein the processor is configured to detect the visual line position based on information obtained by an eye tracker.

12. The endoscope apparatus according to claim 1
wherein the processor is configured to detect the visual line position based on information obtained by a gyro sensor mounted on a head of the operator.

13. A control method of an endoscope apparatus that is connected to an endoscope having an insertion part to be inserted into a living body and an operating part, the method comprising:
displaying an image associated with an operation capable of being executed by the endoscope apparatus on a part of a display screen;
detecting a visual line position of an operator of the endoscope;
determining whether or not the operator gazes at the image based on the detected visual line position; and
executing the operation associated with the image based on a result of the determination and a command signal for giving a command for operation execution, the command signal being input in response to a manipulation by the operator with respect to the operating part.

14. A non-transitory computer readable recording medium storing a control program of an endoscope apparatus that is connected to an endoscope having an insertion part to be inserted into a living body and an operating part, the program causing a computer to execute:
displaying an image associated with an operation capable of being executed by the endoscope apparatus on a part of a display screen;
detecting a visual line position of an operator of the endoscope;
determining whether or not the operator gazes at the image based on the detected visual line position; and
executing the operation associated with the image based on a result of the determination and a command signal for giving a command for operation execution, the command signal being input in response to a manipulation by the operator with respect to the operating part.

15. An endoscope system comprising:
the endoscope apparatus according to claim 1;
the endoscope; and
the display screen.
